(19) United States
(12) Patent Application Publication
(10) Pub. No.: US 2003/0105387 A1
(43) Pub. Date: Jun. 5, 2003

(54) Vaginal speculum and procedure

(76) Inventors: Andrew Frumovitz, Marina del Rey, CA (US); Michael M. Frumovitz, Houston, TX (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/053,014

(22) Filed: Oct. 16, 2001

(51) Int. Cl. A61B 1/32

(52) U.S. Cl. 600/220

(57) ABSTRACT

An improved vaginal speculum including a handle and a dilator in which the dilator is preferably positioned at an angle less than 180 degrees with respect to the handle. The dilator preferably includes a first, shorter dilator member and a second, longer dilator member wherein the shorter dilator member is positioned between the longer member and the handle which may be disposed above the patient during use, thereby facilitating examination of a patient position on a horizontal surface such as a bed.

### BACKGROUND OF THE INVENTION

1. Field of the Invention

The present invention relates to medical devices and, in particular, to an improved vaginal speculum.

2. Description of the Related Art

Vaginal speculums are typically used in medical examinations to dilate the vagina of a patient and thereby provide an effective view of the female reproductive organs. A vaginal speculum 100A of the prior art that has found general use in such examinations is shown in FIG. 1A. Vaginal speculum 100A generally includes a handle 101A and a dilator 102A connected generally perpendicularly to the handle 101A. The dilator 102A includes a first, shorter dilator member 103A, and a second, longer dilator member 104A. The shorter member 103A is typically 10-20 mm shorter than the longer member 104A to facilitate optimal viewing of the cervix. The handle 101A includes a first handle member 122A and a second handle member 124A. The first handle member 122A is connected to the shorter dilator member 103A by a hinge 126A and the second handle member 124A is connected to or integrally formed with the longer dilator member 104A.

A traditional speculum of the type shown in FIG. 1 is typically effective in medical examinations where the patient is positioned at the edge of an exam table with the patient's legs in stirrups. In normal use, the dilator 102A is inserted generally horizontally into the patient's vaginal cavity in a closed position so that the shorter member 103A is in proximity to the upper wall of the vaginal cavity. In light of the fact that the patient is positioned at the edge of the exam table, the practitioner can insert a traditional speculum in the vaginal cavity without interference with the descending handle. The handle members 124A and 122A will then remain outside the patient's body directly below the vaginal opening in a generally vertical relationship with respect to the dilator members 102A and 104A. Thereafter, the shorter member 103A can be adjusted angularly about the hinge 126A by depressing activator 112A descending from the proximal end of the shorter dilator member 103A. In this way, the longer member 104A can be maintained in a substantially fixed horizontal position relative to handle 101A while shorter dilator member 103A can be raised and lowered to obtain the most effective view of the patient's cervix.

In many cases, the patient 105A is examined in less optimal situations, such as on a bed, as shown in FIG. 1A. A bed exam often occurs with gynecological patients who have been hospitalized, obstetrical patients prior to child-birth, and elderly patients who cannot be positioned on the edge of an exam table with legs in stirrups. In such situations, the examination support surface 107A often interferes with the descending handle 101A of speculum 100A at contact point 109A. The handle 101A may also or alternatively interfere with the patient's buttocks. As a result of the interference, practitioners are often unable to tilt the speculum into position for ideal presentation of the cervix. Thus, in order to conduct the examination, the practitioner must typically manipulate the speculum to avoid contact with the examination surface and/or patient's buttocks, thereby causing the patient discomfort. In the process, the touching of the speculum handle with any non-sterile, inanimate object or with the patient's skin can jeopardize the speculum's sterility as well as that of the practitioner's gloved hands.

In order to avoid complications associated with examinations in less optimal situations, practitioners have utilized traditional speculums in an inverted position. Use of a speculum in this alternate arrangement, however, leads to several complications. In inverted use, the longer dilator member 104A is in the upper position adjacent to the cervix and can push the cervix up and behind the top blade during adjustment of the blades, thereby inhibiting effective viewing of the cervix. As a result, the practitioner will continue to adjust the position of the entire speculum in order to avoid contact with the cervix and facilitate viewing in this reversed arrangement. Not only is this alternative arrangement awkward for the patient and practitioner, but often the speculum cannot be properly tilted or manipulated for ideal presentation of the cervix. Moreover, alternative viewing arrangement and manipulation may result in additional discomfort for the patient.

Therefore, there exists a need for an improved vaginal speculum and particularly for an improved vaginal speculum adapted to facilitate use with patients positioned on a horizontal surface, such as a bed, during examination.

### SUMMARY OF THE INVENTION

In accordance with the present invention, there is provided an improved vaginal speculum and particularly an improved vaginal speculum adapted to facilitate examination of a patient positioned on a horizontal examination support surface, such as a bed. The present invention provides an improved speculum that minimizes interference between the handle and matter outside the vaginal cavity and also provides an improved speculum to help reduce the risk of polluting a sterile environment. In addition, the present invention facilitates a reduction in device manipulation during the vaginal examination, thereby improving patient comfort and ease of use.

In a preferred embodiment of the present invention, the vaginal speculum includes a handle and a dilator in which the dilator is preferably positioned at an angle less than 180 degrees with respect to the handle. The dilator preferably includes a first, shorter dilator member and a second, longer dilator member wherein the shorter dilator member is positioned between the longer member and the handle within the angle less than 180 degrees formed between the dilator and the handle.

In another preferred embodiment of the present invention, the handle further includes a first handle member and a second handle member, connected to the dilator members. An actuator is also provided to vary the angular relation of the shorter member with respect to the longer member, thereby facilitating enlargement of the observation opening and providing an optimal view of the cervix. In an alternative embodiment, the actuator may be used to vary the angular relation of the longer member. A coupling device may also be advantageously provided to permit modification of the separation between the first and second handle members. In a preferred embodiment, the second handle member is adapted to move slidably along the first handle member, thereby facilitating adjustment of the spatial relationship between the first and second dilator members.

In accordance with a preferred embodiment of the present invention, the handle is disposed generally perpendicularly to the dilator. In an alternative embodiment, the handle portion is disposed at an angle greater than 90 degrees with respect to the dilator, preferably 100 degrees to 135 degrees, and more preferably at an angle of 110 degrees, thereby advantageously minimizing contact between the handle and the patient's pubis during use.

For a better understanding of the present invention, together with other and further objects thereof, reference is made to the detailed description taken in conjunction with the accompanying drawings and the appended claims.

### BRIEF DESCRIPTION OF THE DRAWINGS

FIG. 1a is a schematic view showing use of a prior art vaginal speculum with a patient disposed on a flat surface, such as a bed.

FIG. 1b is a schematic view showing use of a vaginal speculum in accordance with the present invention with a patient disposed on a flat surface, such as a bed.

FIG. 2 is perspective view of a first embodiment of a vaginal speculum in accordance with the present invention, in which the handle is disposed substantially perpendicular to the dilator and in which the longer dilator member may be advantageously rotated to facilitate examination.

FIG. 3 is a side view of the vaginal speculum of FIG. 2.

FIG. 4 is perspective view of an alternative embodiment of a vaginal speculum in accordance with the present invention, in which the handle is disposed substantially perpendicular to the dilator and in which the shorter dilator member may be advantageously rotated to facilitate examination.

FIG. 5 is a side view of the vaginal speculum of FIG. 4.

FIG. 6 is a side view of another alternative embodiment of a vaginal speculum in accordance with the present invention, in which the handle is disposed at an obtuse angle with respect to the dilator, whereby contact between the handle and the patient's pubis is advantageously minimized during use.

### DETAILED DESCRIPTION OF THE PREFERRED EMBODIMENTS

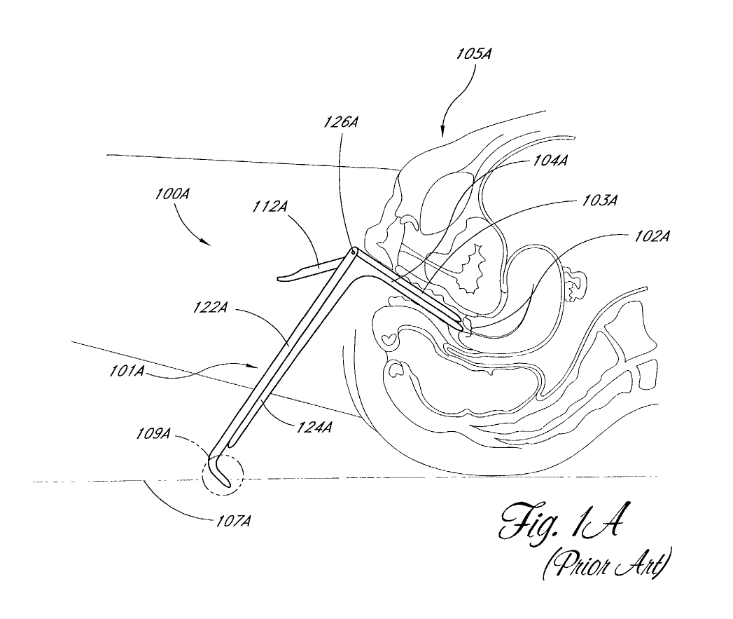

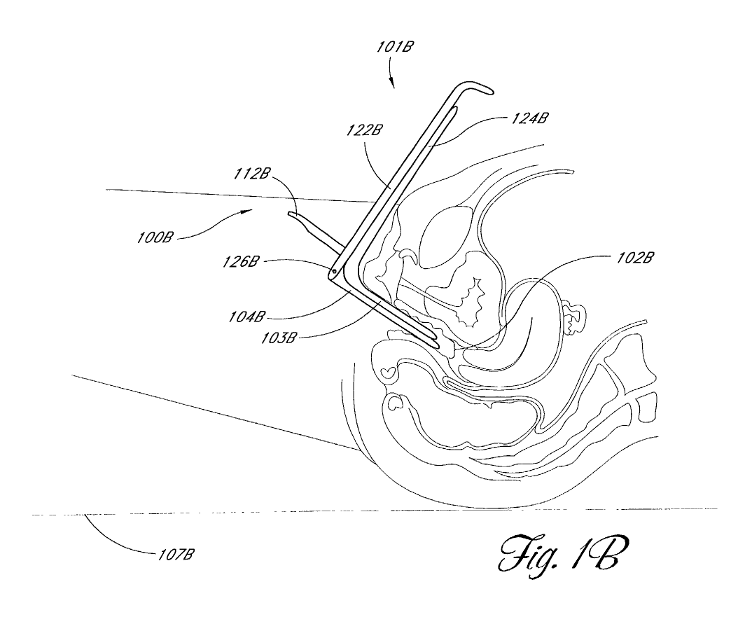

With reference now to the exemplary drawings and particularly FIG. 1B, there is shown an improved vaginal speculum in accordance with the present invention which facilitates an effective view of the female reproductive organs during examination while minimizing interference with external objects or the patient and improving patient comfort. Vaginal speculum 100B generally includes a handle 101B and a dilator 102B connected generally perpendicularly to the handle 101B. The dilator 102B includes a first, shorter dilator member 103B, and a second, longer dilator member 104B. The shorter member 103B is typically 10-20 mm shorter than the longer member 104B to facilitate optimal viewing of the cervix. The handle 101B includes a first handle member 122B and a second handle member 124B.

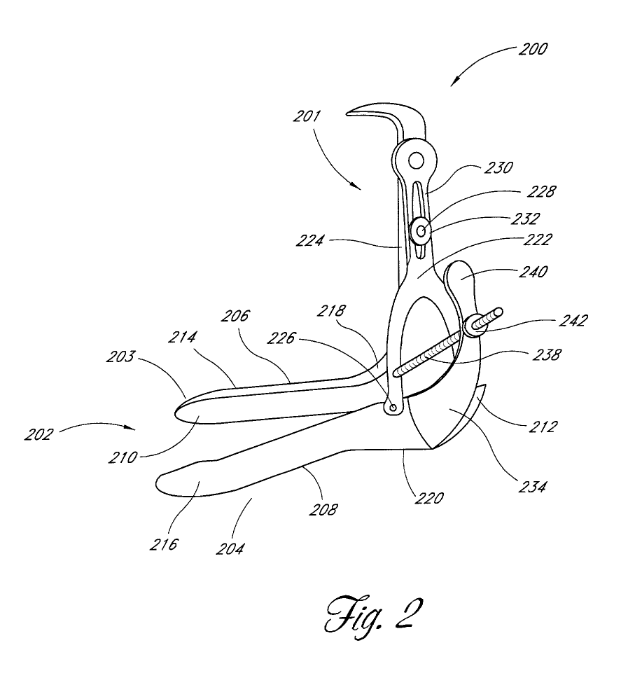

During examination, the dilator 102B is inserted into the patient's vaginal cavity in a closed position so that the shorter member 103B is in proximity to the upper wall of the vaginal cavity and the handle is disposed generally vertically, above the patient's vaginal opening. In light of the fact that the handle is positioned above the patient, the practitioner can insert the speculum 100B in the vaginal cavity without interference with the examination support surface 107B or the patient's buttocks. In a preferred embodiment, the shorter dilator member 103B can be adjusted angularly by depressing activator 112B descending from the proximal end of the dilator 102B. In this way, the longer member 104A can be maintained in a substantially fixed horizontal position relative to handle 101A while shorter dilator member 103A can be raised and lowered to obtain the most effective view of the patient's cervix. As an alternative, the shorter member 103B can be maintained in a substantially fixed horizontal position relative to handle 101B while longer dilator member 104B could be adjusted angularly using activator 112B. These embodiments are discussed in more detail in connection with the detailed description accompanying FIGS. 2-6, below.

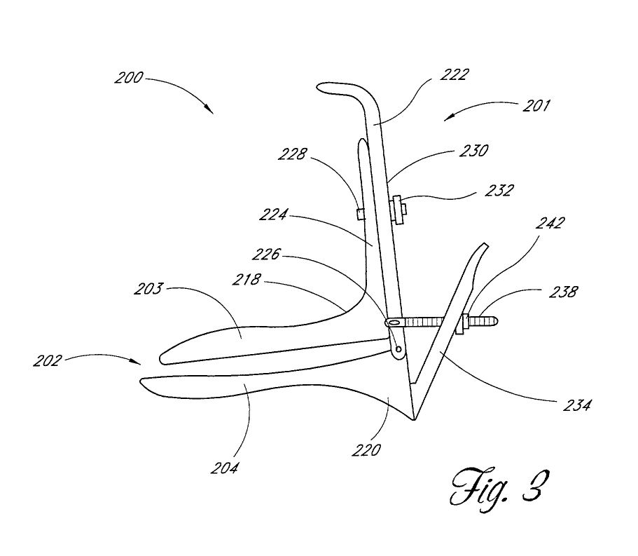

Referring now to FIGS. 2 and 3, there is illustrated a vaginal speculum 200 having a handle portion 201 disposed generally perpendicularly to a dilator portion 202. Dilator portion 202 includes a first, shorter dilator member 203 and a second, longer dilator member 204. The shorter member 203 is advantageously positioned between the handle portion 201 and the second, longer member 204. The difference in length between the two members 203, 204 is preferably about 10-20 mm. Handle portion 201 includes a first handle member 222 and a second handle member 224. First handle member 224 is preferably wishbone-shaped to provide an adequate view of the cervix during use.

Dilator members 203, 204 each have an outer convex surface 206, 208 and an inner concave surface 210, 212 as illustrated in FIG. 2. Dilator members 203, 204 each include respective blade portions 214, 216, which are preferably arcuate in cross-section. Each dilator member 203, 204 also includes an elongated neck 218, 220 proximate the handle portion 201 adapted to push the patient's tissue aside during use and thereby reduce the possibility of pinching.

An apparatus is advantageously provided so that the relative separation between dilation members 203 and 204 can be varied and to optionally lock or maintain the dilation members in a selected relative orientation. As illustrated in FIG. 3, the dilator members 203 and 204 extend generally parallel when slightly spaced apart or, alternatively, when in the closed position. A screw 228 is mounted to the first handle member 222. The screw 228 extends through a slot 230 in second handle member 224 and is engaged by a knurled nut 232 to form a slidable connection between first handle member 222 and second handle member 224. Knurled nut 232 can be loosened so that the position of the first and second handle members 222 and 224 can be varied, thereby facilitating corresponding variations in the spacing between dilator members 203 and 204. The knurled nut 232 can be tightened to engage the portion of first handle member 232 surrounding slot 230 adjacent to screw 238 in order to maintain the relative position of the first and second handle portions 222 and 224 and, thus, the relative spacing of the first and second dilator members 203 and 204.

As shown in the embodiment in FIGS. 2 and 3, the vaginal speculum further includes an apparatus for varying the relative orientation of the first dilator member 203 with respect to the second dilator member 204. In this embodiment, the first handle member 222 is coupled to dilator member 204 by a hinge, formed by the combination of pivot pins 226 and openings in the ends of first handle member 222. Meanwhile, the second handle member 224 is optionally integrally formed with first dilator member 203. An actuator 234 is provided, which may be integrally formed with the second dilator member 204 and which extends from a segment of the second dilator member 204 proximate to handle 201. By pressing and releasing actuator 234, second, longer dilator member 204 may be pivotally rotated relative to the handle 201 and first, shorter dilator member at the hinge.

The vaginal speculum of FIGS. 2 and 3 further includes an actuator lock generally formed by screw 238 and positioning nut 242. As shown in FIG. 3, screw 238 is attached to the second handle member 224 and extends through an aperture in actuator 234 and is engaged by a positioning nut 242, thereby releasably restraining longer member 204 in its adjusted spatial relationship with respect to shorter member 203.

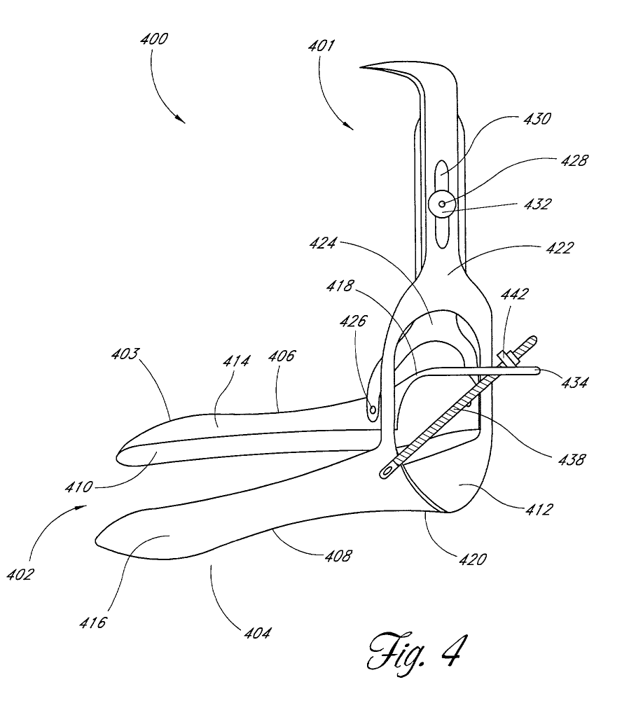

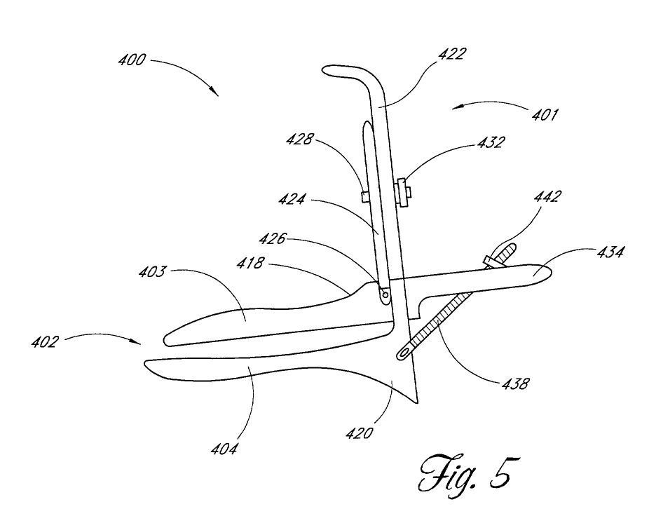

Referring to an alternative embodiment of the present invention shown in FIGS. 4 and 5, there is illustrated a vaginal speculum 400 having a handle portion 401 disposed generally perpendicularly to a dilator portion 402. Dilator portion 402 includes a first, shorter dilator member 403 and a second, longer dilator member 404. The shorter dilator member 403 is advantageously positioned between the handle portion 401 and the second, longer member 404. The difference in length between the two members 403, 404 is preferably about 10-20 mm. Handle portion 401 includes a first handle member 422 and a second handle member 424. First handle member 422 is preferably wishbone-shaped to provide an adequate view of the cervix during use.

Dilator members 403, 404 each have an outer convex surface 406, 408 and an inner concave surface 410, 412 as illustrated in FIG. 4. Dilator members 403, 404 each include a respective blade portion 414, 416, which are arcuate in cross-section. Each dilator member 403, 404 also includes an elongated neck 418, 420 proximate the handle portion 401 adapted to push the patient's tissue aside during use and thereby reduce the possibility of pinching.

An apparatus is advantageously provided so that the relative separation between dilation members 403 and 404 can be varied and to optionally lock or maintain the dilation members in a selected relative orientation. As illustrated in FIG. 4, the dilator members 403 and 404 extend generally parallel when in slightly spaced apart or, alternatively, in the closed position. A screw 428 is mounted to the first handle member 422. The screw 428 extends through a slot 430 in second handle member 424 and is engaged by a knurled nut 432 to form a slidable connection between first handle member 422 and second handle member 424. Knurled nut 432 can be loosened so that the position of the first and second handle members 422 and 424 can be varied, thereby facilitating corresponding variations in the spacing between dilator members 403 and 404. The knurled nut 432 can be tightened to engage the portion of first handle member 432 surrounding slot 430 adjacent to screw 438 in order to maintain the relative position of the first and second handle portions 422 and 424 and, thus, the relative spacing of the first and second dilator members 403 and 404.

As shown in the embodiment in FIGS. 4 and 5, the vaginal speculum further includes an apparatus for varying the relative orientation of the first dilator member 403 with respect to the second dilator member 404. In this embodiment, the first handle member 422 is coupled to first, shorter dilator member 403 by a hinge, formed by the combination of pivot pins 426 and openings in the ends of first handle member 422. Meanwhile, the second handle member 424 is optionally integrally formed with second, longer dilator member 404. An actuator 434 is provided, which may be integrally formed with the first, shorter dilator member 403 and which extends from a segment of the first dilator member 403 proximate to handle 401. By pressing and releasing actuator 434, the first dilator member 403 may be pivotally rotated relative to the handle 401 and second, longer dilator member 404 at the hinge.

The vaginal speculum of FIGS. 4 and 5 further includes an actuator lock generally formed by screw 438 and positioning nut 442. As shown in FIG. 5, screw 438 is attached to the first handle member 424 and extends through an aperture in actuator 434 and is engaged by a positioning nut 442, thereby releasably restraining longer member 404 in its adjusted spatial relationship with respect to shorter member 403.

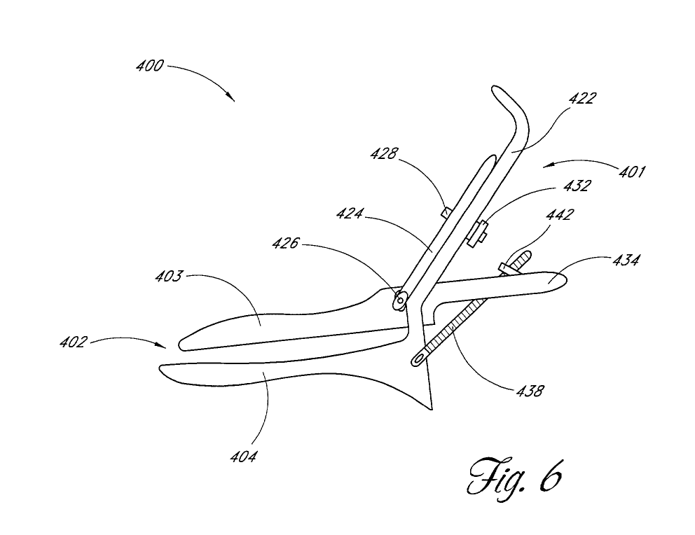

Referring now to FIG. 6, an alternative embodiment of the vaginal speculum shown in FIGS. 4 and 5 is illustrated. As with the embodiment shown in FIGS. 4 and 5, vaginal speculum 400 includes a handle portion 401 and dilator portion 402. In this alternative embodiment, however, the handle portion 401 is disposed at an obtuse angle to the dilator portion in order to minimize or eliminate contact with the patient's pubis during use. In the embodiment illustrated in FIG. 6, the handle portion is disposed at a preferred angle of about 110 degrees. In accordance with the present invention, it will be appreciated that the handle may be advantageously disposed at an obtuse angle, preferably between about 100 degrees and 135 degrees.

As with the embodiment shown in FIGS. 4 and 5, the vaginal speculum of FIG. 6 includes an apparatus to facilitate variation and locking of the relative separation between dilation members 403 and 404 and an apparatus for varying the relative orientation of the first dilator member. It will be appreciated that like reference numerals are used to identify like elements.

Specula in accordance with the present invention are preferably made of stainless steel. It is anticipated, however, that a variety of materials, such as rigid plastic, may be utilized advantageously. It will be appreciated by those of ordinary skill in the art that the preferred materials for construction of the speculum will be substantially rigid and conveniently sterilized or discarded.

In operation of the embodiment shown in FIGS. 2 and 3, the practitioner grips handle members 222 and 224 and inserts shorter member 203 and longer member 204 (in a closed position) into the patient's vaginal cavity. The members 203, 204 are preferably placed in an effective position for viewing the cervix and the separation between shorter dilator member 203 and longer dilator member 204 adjusted and locked by adjusting knurled nut 232. By depressing actuator 234, the practitioner can then adjust the angle of the longer dilator member 204 to provide further separation between the distal ends of the shorter member 203 and longer member 204. This distends the walls of the vagina so that the reproductive organs are more readily visible. The angle of the longer member 204 may then be locked in place by adjusting location of positioning nut 242 along screw 238. Alternatively, using the embodiment of the present invention shown in FIGS. 4 and 5, the practitioner can adjust the angle of the shorter dilator member 403 by depressing the actuator 434, whereby the angle of shorter dilator member 403 is adjusted to provide further separation between the distal ends of the shorter member 403 and longer member 404. The angle of the shorter member 403 may then be advantageously locked in place using positioning nut 442.

Following examination, the vaginal speculum shown in FIGS. 2 and 3 may be removed. In doing so, the locking nut 232 and positioning nut 240 are preferably loosened and the shorter dilator member 203 and longer dilator member 204 brought together in the closed position. Using handle portion 201, the dilator portion 202 may be removed from the vaginal cavity. Throughout this process, the handle portion 201 remains positioned above dilator 202 away from the horizontal surface on which the patient is disposed and away from the patient's buttocks. The vaginal speculum of FIGS. 4 and 5 may be removed in a similar manner.

Although the present invention has been described in terms of certain preferred embodiments, those skilled in the art will recognize that other and further changes and modifications may be made hereto without departing from the spirit of the invention, and it is intended to claim all such changes and modifications as fall within the true scope of the invention. Accordingly, the scope of the present invention is not to be limited by the particular embodiments described, but is to be defined only by reference to the appended claims and equivalents thereof

What is claimed is:

1. A vaginal speculum comprising:
a handle; and
a dilator comprising a first, shorter dilator member and a second, longer dilator member;
wherein the shorter dilator member is positioned between the longer member and the handle.

2. The vaginal speculum of claim 1, further comprising an actuator adapted to rotate at least one dilator member.

3. The vaginal speculum of claim 2, wherein the actuator is adapted to rotate the shorter dilator member.

4. The vaginal speculum of claim 2, wherein the actuator is adapted to rotate the longer dilator member.

5. The vaginal speculum of claim 1, wherein the second, longer member is pivotable proximate to the junction of the second dilator member and the handle.

6. The vaginal speculum of claim 1, wherein the first, shorter member is pivotable proximate to the junction of the first dilator member and the handle.

7. The vaginal speculum of claim 1, wherein the dilator is disposed at an angle substantially 90 degrees with respect to the handle.

8. The vaginal speculum of claim 1, wherein the dilator is disposed at an obtuse angle with respect to the handle.

9. The vaginal speculum of claim 1, wherein the dilator is disposed at an angle between about 100 degrees and 145 degrees with respect to the handle.

10. The vaginal speculum of claim 1, wherein the dilator is disposed at an angle between about 90 degrees and 135 degrees with respect to the handle.

11. The vaginal speculum of claim 1, wherein the first and second members comprise an arcuate blade.

12. The vaginal speculum of claim 1, wherein the handle comprises a first handle member and a second handle member.

13. The vaginal speculum of claim 11, wherein the first handle member is integrally formed to the longer member.

14. The vaginal speculum of claim 11, wherein the first handle member is pivotably attached to the longer dilator member.

15. The vaginal speculum of claim 11, wherein the second handle member is integrally formed to the shorter dilator member.

16. The vaginal speculum of claim 11, wherein the second handle member is pivotably attached to the shorter dilator member.

17. The vaginal speculum of claim 1, further comprising a coupler adapted to facilitate adjustment of the relative spatial position between the first and second dilator members.

18. a first, shorter dilator member;
a second, longer dilator member;
an actuator adapted to pivotally rotate;
a handle comprising a first handle member and a second handle member, wherein the first handle member is attached to the longer dilator member and the second handle member is intregally formed with the shorter dilator member;
wherein the shorter dilator member and the longer dilator member are positioned within an angle less than 180 degrees of the handle, and wherein the shorter dilator member is positioned between the handle and the longer member and within the angle less than 180 degrees.

19. The vaginal speculum of claim 17, further comprising a coupling device adapted to slidably adjust the relative spatial position between the first and second dilator members.

20. A vaginal speculum comprising:
a first, longer dilator member;
a second, shorter dilator member;
an actuator adapted to pivotally rotate the shorter member with respect to the longer member;
a handle comprising a first handle member and second handle member, wherein the first handle member is integrally formed with the longer dilator member and the second handle member is attached to the shorter dilator member;
wherein the shorter dilator member and the longer dilator member are positioned within an angle less than 180 degrees of the handle, and wherein the shorter dilator member is positioned between the handle and the longer dilator member and within the angle less than 180 degrees.

21. The vaginal speculum of claim 19, further comprising a coupling device for slidably adjusting the relative spatial position between the first and second dilator members.

22. A method for examining a patient lying on her back, comprising:
positioning a speculum in a vaginal cavity using a handle disposed above a dilator having a shorter member and a longer member, wherein the longer member is below the shorter member.

23. The method of claim 21, further comprising sliding the handle upward to create a relative spatial relation between the dilator members.

24. The method of claim 21, further comprising pivoting the shorter member with respect to the longer member to adjust the angular relation between the members.

25. The method of claim 21, further comprising pivoting the longer member with respect to the shorter member to adjust the angular relation between the members.
